(19) United States
(12) Patent Application Publication
(10) Pub. No.: US 2021/0277105 A1
(43) Pub. Date: Sep. 9, 2021

(54) TREATING ULCERATIVE COLITIS WITH BRAZIKUMAB

(71) Applicant: ASTRAZENECA COLLABORATION VENTURES, LLC, Wilmington, DE (US)

(72) Inventors: Carl Gommoll, Robbinsville, NJ (US); Aparna Sahoo, Princeton, NJ (US)

(21) Appl. No.: 17/259,448

(22) PCT Filed: Jul. 11, 2019
(86) PCT No.: PCT/IB19/00720

§ 371 Date: Jan. 11, 2021

Related U.S. Application Data
(60) Provisional application No. 62/697,939, filed on Jul. 13, 2018.

(51) Int. Cl. C07K 16/24; A61K 9/00; A61P 1/04

(52) U.S. Cl. CPC C07K 16/244; A61P 1/04; A61K 9/0019

(57) ABSTRACT

The disclosure relates to products and methods for treating ulcerative colitis. The products relate to antibodies that inhibit native human IL-23, but do not inhibit IL-12.

### CROSS-REFERENCE TO RELATED APPLICATIONS

This application is claims priority to Provisional U.S. Patent Application No. 62/697,939, filed Jul. 13, 2018, which is hereby incorporated by reference in its entirety.

### INCORPORATION BY REFERENCE OF MATERIALS SUBMITTED ELECTRONICALLY

This application contains, as a separate part of disclosure, a Sequence Listing in computer readable form (Filename: 53230A_Seqlisting.txt; Size: 4,764 bytes; Created: Jul. 11, 2019), which is incorporated herein by reference in its entirety.

### FIELD

The disclosure relates to products and methods for treating ulcerative colitis. The products relate to antibodies that inhibit native human IL-23 but do not inhibit IL-12.

### BACKGROUND

Ulcerative colitis (UC) is an idiopathic, chronic inflammatory disorder of the colonic mucosa, which starts in the rectum and generally extends proximally in a continuous manner through part of, or the entire, colon. Bloody diarrhea is the characteristic symptom of the disease, with prominent symptoms of rectal urgency and tenesmus. The clinical course is unpredictable, marked by alternating periods of exacerbation and remission, which may occur spontaneously or in response to treatment. The precise cause of inflammatory bowel disease (IBD) is unknown; however, genetically susceptible individuals seem to have a dysregulated mucosal immune response to commensal gut flora, which results in bowel inflammation. North America and northern Europe have the highest incidence and prevalence rates of UC, with incidence varying from 9 to 20 cases per 100,000 person-years, and prevalence rates from 156 to 291 cases per 100,000 people, with similar prevalence among males and females. UC has a bimodal pattern of incidence, with the main onset peak between ages 15 and 30 years, and a second smaller peak between ages 50 and 70 years. It is currently estimated that there are approximately 800,000 people afflicted with UC in the United States and 1.4 million in Europe. Some patients may have persistent clinically active disease. The current treatment options for patients with moderate to severe UC that is refractory to standard therapies are limited. Those standard therapies include 5-aminosalicylates, glucocorticosteroids, 6-mercaptopurine, azathioprine, methotrexate, anti-tumor necrosis factor alpha (TNFα) monoclonal antibodies, and vedolizumab.

IL-23, a member of the IL-12 family of cytokines, is a heterodimeric cytokine that potently induces pro-inflammatory cytokines. IL-23 is related to the heterodimeric cytokine Interleukin 12 (IL-12) both sharing a common p40 subunit. In IL-23, a unique p19 subunit is covalently bound to the p40 subunit. In IL-12, the unique subunit is p35 (Oppmann et al., Immunity, 2000, 13: 713-715). IL-23 is expressed by antigen presenting cells (such as dendritic cells and macrophages) in response to activation stimuli such as CD40 ligation, Toll-like receptor agonists and pathogens. IL-23 binds a heterodimeric receptor comprising an IL-12Rβ1 subunit (which is shared with the IL-12 receptor) and a unique receptor subunit, IL-23R.

IL-23 acts on activated and memory T cells and promotes survival and expansion of the T cell subset, Th17. Th17 cells produce proinflammatory cytokines, including IL-6, IL-17, TNFα, IL-22 and GM-CSF. IL-23 also acts on natural killer cells, dendritic cells and macrophages to induce pro-inflammatory cytokine expression. Unlike IL-23, IL-12 induces the differentiation of naïve CD4+ T cells into mature Th1 IFNγ-producing effector cells, and induces NK and cytotoxic T cell function by stimulating IFNγ production. Th1 cells driven by IL-12 were previously thought to be the pathogenic T cell subset in many autoimmune diseases; however, more recent animal studies in models of inflammatory bowel disease, psoriasis, inflammatory arthritis and multiple sclerosis, in which the individual contributions of IL-12 and IL-23 were evaluated, have firmly established that IL-23, not IL-12, is the key driver in autoimmune/inflammatory disease (Ahern et al., Immun. Rev. 2008 226:147-159; Cua et al., Nature 2003 421:744-748; Yago et al., Arthritis Res and Ther. 2007 9(5): R96). It is believed that IL-12 plays a critical role in the development of protective innate and adaptive immune responses to many intracellular pathogens and viruses and in tumor immune surveillance. See Kastelein, et al., Annual Review of Immunology, 2007, 25: 221-42; Liu, et al., Rheumatology, 2007, 46(8): 1266-73; Bowman et al., Current Opinion in Infectious Diseases, 2006 19:245-52; Fieschi and Casanova, Eur. J. Immunol. 2003 33:1461-4; Meeran et al., Mol. Cancer Ther. 2006 5: 825-32; Langowski et al., Nature 2006 442: 461-5. As such, IL-23 specific inhibition (sparing IL-12 or the shared p40 subunit) is expected to have a superior safety profile compared to dual inhibition of IL-12 and IL-23.

In view of the foregoing observations, it is apparent that that there is a need for new modalities for the treatment of ulcerative colitis that specifically target IL-23 without the potential risks associated with inhibition of IL-12.

### SUMMARY

Disclosed herein is an IL-23 blockade that provides a mechanism to inhibit the inflammation and reduce clinical symptoms associated with ulcerative colitis (UC). The IL-23 blockade specifically inhibits IL-23 and does not inhibit IL-12, i.e., results in minimal (less than 1% IL-12 inhibition) or no inhibition of IL-12 activity following administration of brazikumab. In some embodiments, the IL-23 blockade specifically inhibits IL-23 and there is no inhibition of IL-12. Specifically targeting IL-23 with brazikumab is expected to offer a better benefit:risk profile compared with IL-12/23 antibodies.

In one aspect, the disclosure provides a method of treating ulcerative colitis in a subject comprising administering a therapeutically effective amount of an anti-IL-23 antibody that does not inhibit IL-12 to a subject with ulcerative colitis. In some embodiments of the method, the subject has moderately to severely active ulcerative colitis as determined by clinical features, colonoscopy, and/or histological findings. In some embodiments, the anti-IL-23 antibody is administered by intravenous infusion such as by administering induction dosages of at least 700, at least 720, at least 1400, at least 1440, at least 2100, at least 2180, or at least 4200 mg of anti-IL-23 antibody, typically in a volume of about 100 ml. In some embodiments, the intravenous infusion comprises at least 70 mg of anti-IL-23 antibody in a volume of about 100 ml delivered over a period of at least 30 minutes, e.g., at least 60 minutes. Embodiments are also contemplated wherein the intravenous infusion further comprises a pharmaceutically acceptable adjuvant, diluent or carrier, which may include 5% (w/v) dextrose.

Embodiments of the method are also contemplated wherein a plurality of intravenous infusions is administered. In some embodiments, the plurality of intravenous infusions each comprise the same quantity of anti-IL-23 antibody.

Embodiments of the disclosure also exist wherein the anti-IL-23 antibody is administered subcutaneously. In some of these embodiments, the anti-IL-23 antibody is administered in a plurality of doses. In some embodiments, a total dosage of at least 105 or at least 210 mg of anti-IL-23 antibody is administered. In some embodiments, each dose comprises about 70 mg anti-IL-23 antibody. In some embodiments, a total dosage of at least 120 mg or at least 240 mg is administered, e.g., subcutaneously, with each dose comprising about 120 mg of anti-IL23 antibody.

Also contemplated are embodiments of the method further comprising a plurality of doses of anti-IL-23 antibody, wherein a second dose is administered about two weeks after a first dose, and a third and subsequent doses are administered about four weeks after a preceding dose. In some embodiments, the plurality of doses is about 10 doses. In some embodiments, the first and second doses, or the first three doses, are administered by intravenous infusion and any subsequent dose is administered subcutaneously. In some embodiments, each dose comprises at least 70 mg of anti-IL-23 antibody. In some embodiments, each dose comprises at least 120 mg of anti-IL-23 antibody.

The methods of the disclosure may further comprise measuring the effect of the therapy using the modified Mayo Score/Disease Activity Index for Ulcerative Colitis. In some embodiments, the therapy lowers the score of at least two components of the modified Mayo Score/Disease Activity Index for Ulcerative Colitis, wherein the components are selected from the group consisting of Stool Frequency, Rectal Bleeding, Endoscopy Findings and Physicians' Global Assessment.

The disclosure also includes methods, as described herein, wherein the anti-IL-23 antibody comprises the CDRH1 of SEQ ID NO:3, the CDRH2 of SEQ ID NO:4, the CDRH3 of SEQ ID NO:5, the CDRL1 of SEQ ID NO:6, the CDRL2 of SEQ ID NO:7 and the CDRL3 of SEQ ID NO:8. In some embodiments, the anti-IL-23 antibody comprises the heavy chain variable region sequence of SEQ ID NO:1. In some embodiments, the anti-IL-23 antibody comprises the light chain variable region sequence of SEQ ID NO:2. In some embodiments, the anti-IL-23 antibody comprises the heavy chain variable region sequence of SEQ ID NO:1 and the light chain variable region sequence of SEQ ID NO:2.

Other features and advantages of the disclosure will become apparent from the following detailed description, including the drawing. It should be understood, however, that the detailed description and the specific examples, while indicating embodiments, are provided for illustration only, because various changes and modifications within the spirit and scope of the disclosure will become apparent to those skilled in the art from the detailed description.

### BRIEF DESCRIPTION OF THE DRAWINGS

FIG. 1 presents the results of the pharmacokinetic analysis of an ascending single dose study of subcutaneous administration of AMG 139 (i.e., brazikumab) in healthy subjects (HS). The results shown illustrate the mean (±SD) serum AMG 139 concentration-time profiles.

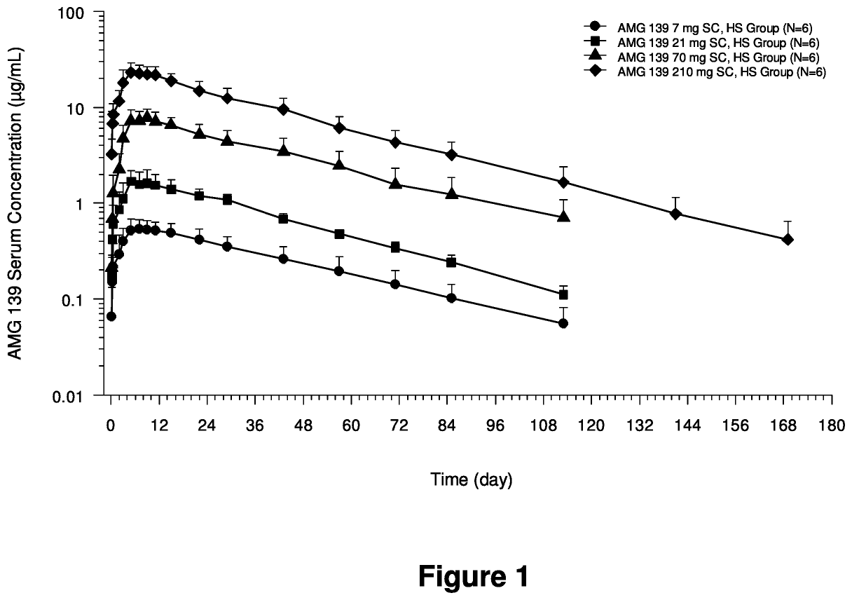

FIG. 2 presents the results of the pharmacokinetic analysis of an ascending single dose study of intravenous administration of AMG 139 in healthy subjects (HS). The results shown illustrate the mean (±SD) serum AMG 139 concentration-time profiles.

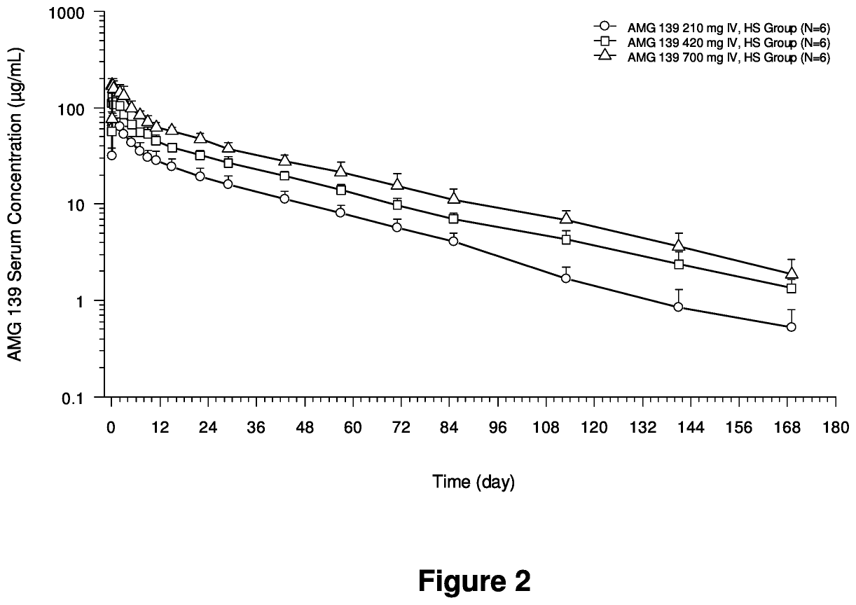

FIG. 3 presents the pharmacokinetic structural model used in developing the AMG 139 quantitative population PK model based on data from Example 1.

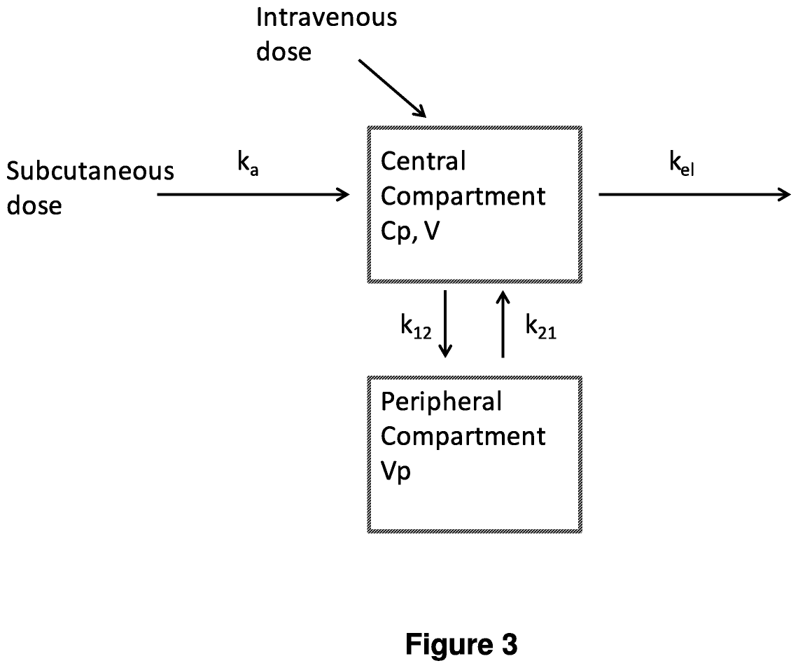

FIG. 4 presents the results of a diagnostic visual predictive check of the AMG 139 population PK model. The results shown illustrate the mean (solid line) and 90% confidence interval (dashed line) AMG 139 concentration-time profile after simulating 1000 clinical trials. Each point represents actual, observed concentrations from subjects.

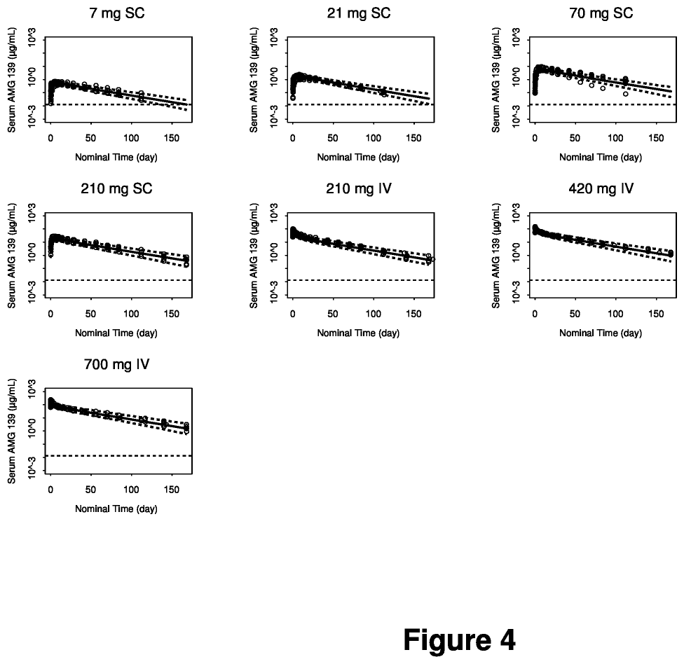

FIG. 5 presents the results of multiple diagnostic visual predictive checks of the AMG 139 population PK model. The results illustrate correlations between observed AMG 139 concentrations and that of population and individual predicted concentrations, as well as the weighted residuals of model fitting between population predicted concentrations and time.

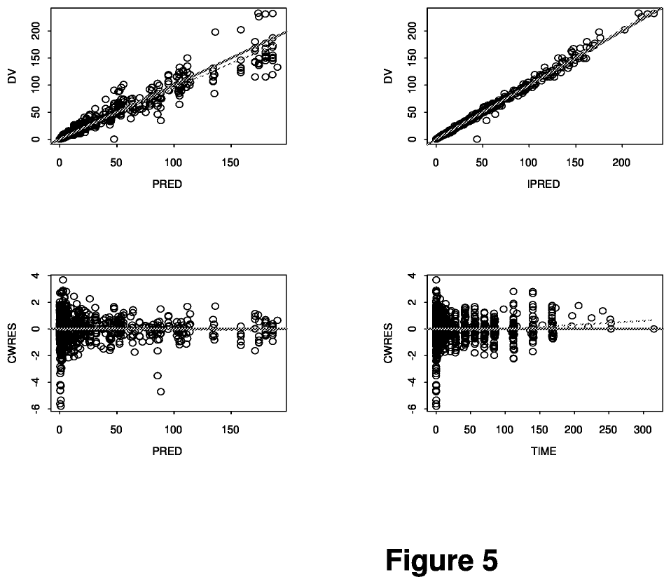

FIG. 6 presents the amino acid sequences of brazikumab heavy and light chain variable regions, which are presented as SEQ ID NOs:1 and 2, respectively. Underscored amino acid sequences identify the six complementarity determining regions, i.e., CDRH1 (SEQ ID NO:3), CDRH2 (SEQ ID NO:4), CDRH3 (SEQ ID NO:5), CDRL1 (SEQ ID NO:6), CDRL2 (SEQ ID NO:7), and CDRL3 (SEQ ID NO:8).

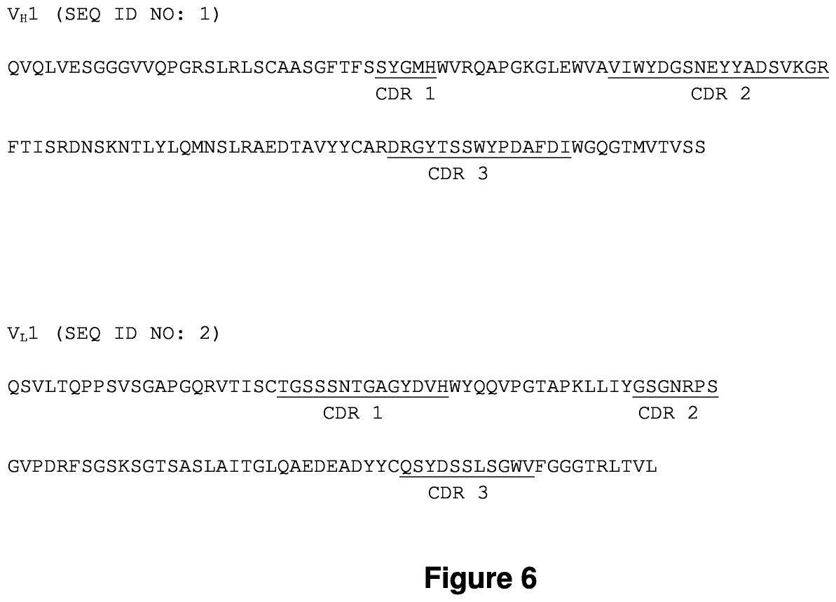

### DETAILED DESCRIPTION

The disclosure provides methods of treating, including ameliorating a symptom of, ulcerative colitis by administering an effective amount of an anti-IL-23 antibody that inhibits an activity of IL-23 without inhibiting the activity of IL-12. The anti-IL-23 antibodies of the disclosure include all known forms of antibodies, provided that those antibody forms specifically bind and inhibit IL-23 without affecting the activity of IL-12. It is contemplated that the methods of the disclosure are well-suited for the treatment of patients with moderately to severely active ulcerative colitis, typically as judged by a skilled clinician interpreting the results of a colonoscopy. The disclosed methods provide a cost-effective approach to bringing beneficial relief to those suffering from ulcerative colitis.

The terms “treating”, and “treatment” and the like are used herein to generally mean obtaining a desired pharmacological, physiological or therapeutic effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease and/or its symptoms or conditions. The disclosure is directed towards treating a patient suffering from disease related to pathological inflammation. The present disclosure provides materials and methods for preventing, inhibiting, or relieving adverse effects attributed to pathological inflammation over long periods of time and/or are such as are caused by physiological response to inappropriate inflammation present in a biological system over a long period of time.

An anti-IL-23 antibody that does not inhibit IL-12, as used herein, means an anti-IL-23 antibody that results in minimal to no inhibition of IL-12 activity. An upper bound on minimal inhibition of IL-12 activity is less than 1% inhibition of IL-12 activity following administration of brazikumab.

In one aspect, the present disclosure provides methods of treating a subject. The method can, for example, have a generally beneficial effect on the subject, e.g., it can increase the subject's expected longevity. Alternatively, the method can, for example, treat, prevent, cure, relieve, or ameliorate (“treat”) a disease, disorder, condition, or illness (“a condition”). In one embodiment, the disclosure provides a method of treating a condition in a subject comprising administering the pharmaceutical composition comprising an IL-23-specific antibody to the subject, wherein the condition is treatable by reducing the activity (partially or fully) of IL-23 in the subject. Treating encompasses both therapeutic administration (i.e., administration when signs and symptoms of the disease or condition are apparent) as well prophylactic or maintenance therapy (i.e., administration when the disease or condition is quiescent), as well as treating to induce remission and/or maintain remission. Accordingly, the severity of the disease or condition can be reduced (partially, significantly or completely), or the signs and symptoms can be prevented or delayed (delayed onset, prolonged remission, or quiescence).

Among the conditions to be treated in accordance with the present disclosure are conditions in which IL-23 is associated with or plays a role in contributing to the underlying disease or disorder or otherwise contributes to a negative symptom. Such conditions include bowel inflammation, such as that characterizing ulcerative colitis.

The term “efficacy” as used herein in the context of a dosage regimen refers to the effectiveness of a particular treatment regimen. Efficacy can be measured based on change in the course of a disease in response to an agent of the present disclosure. In one embodiment, an antigen-binding protein (for example, an anti-IL-23 antibody) is administered to the subject in an amount and for a time sufficient to induce an improvement, preferably a sustained improvement, in at least one indicator that reflects the severity of the disorder that is being treated. Various indicators that reflect the extent of the subject's illness, disease or condition may be assessed for determining whether the amount and time of the treatment is sufficient. Such indicators include, for example, clinically recognized indicators of disease severity, symptoms, or manifestations of the disorder in question.

In one embodiment, an improvement is considered to be sustained if the subject exhibits the improvement on at least two occasions separated by two to four weeks. In another embodiment, an improvement is considered to be sustained if the subject exhibits the improvement on at least two occasions separated by two to four months; in a further embodiment, an improvement is considered to be sustained if the subject exhibits the improvement on at least two occasions separated by six to twelve months. The degree of improvement generally is determined by a physician, who may make this determination based on signs, symptoms, colonoscopies, biopsies, or other test results, and who may also employ questionnaires that are administered to the subject, such as quality-of-life questionnaires developed for a given disease such as ulcerative colitis.

The IL-23 specific antibody may be administered to achieve an improvement in a subject's condition. Improvement may be indicated by a decrease in an index of disease activity, by amelioration of clinical symptoms, endoscopic improvement, or by any other measure of disease activity.

Treatment of a subject with an IL-23 specific antibody may be given in an amount and/or at sufficient interval to achieve and/or maintain a certain quantity of IL-23-specific antibody per volume of serum using, for example, an assay as described herein. For example, the heterodimer specific antibody is given to achieve a serum concentration of 12.5 ng/ml to 1000 ng/ml. In one embodiment, the heterodimer-specific antibody is given to achieve a serum concentration of at least 12.5 ng/ml, 25 ng/ml, 50 ng/ml, 60 ng/ml, 70 ng/ml, 75 ng/ml, 80 ng/ml, 85 ng/ml, 90 ng/ml, 95 ng/ml, 100 ng/ml, 150 ng/ml, 200 ng/ml, 500 ng/ml, or 990 ng/ml. Those of skill in the art will understand that the amounts given here apply to a full-length antibody or immunoglobulin molecule; if an antigen-binding fragment thereof is used, the same molar serum concentration can be achieved although the weight per unit volume will differ from that given in a manner that can be calculated based on the molecular weight of the fragment and the full-length immunoglobulin.

Treatment of a subject with an IL-23-specific antibody may be given in an amount and at an interval of 15-54 mg every 0.5-1.5 months; 55-149 mg every 1.5-4.5 months; 150-299 mg every 4-8 months; or 300-1100 mg every 14-8 months. In one embodiment the amount and interval are selected from the group consisting of: 21 mg every month; 70 mg every 3 months; 210 mg every 6 months; or 700 mg every 6 months.

It is understood that the methods of treating the diseases described herein would administer an effective amount of an anti-IL-23 antibody. Depending on the indication to be treated, a therapeutically effective amount is sufficient to cause a reduction in at least one symptom of the targeted pathological condition by at least about 5%, 10%, 15%, 20%, 25%, 30%, 35%, 40%, 45%, 50%, 55%, 60%, 65%, 70%, 75%, 80%, 85%, 90%, 95%, or more, relative to untreated subjects.

Administration and dosage regimens of an anti-IL-23 antibody can be adjusted to provide an effective amount for an optimum therapeutic response. For example, a single bolus can be administered, several divided doses can be administered over time or the dose can be proportionally reduced or increased as indicated by the exigencies of the therapeutic situation. The anti-IL-23 antibody may be administered by any suitable technique, including but not limited to, parenterally, topically, or by inhalation. If injected, the pharmaceutical composition can be administered, for example, via intra-articular, intravenous, intramuscular, intralesional, intraperitoneal or cutaneous routes (including intra-, trans- or sub-dermal, and subcutaneous), by bolus injection, or continuous infusion. In some embodiments, the pharmaceutical composition is administered by an intravenous route. In some embodiments the pharmaceutical composition is administered by a subcutaneous route. In further embodiments, the compositions are administered by oral, buccal, rectal, intratracheal, gastric, or intracranial routes. Localized administration, e.g., at a site of disease or injury is contemplated, for example, by enema or suppository for conditions involving the gastrointestinal tract. Also contemplated are transdermal delivery and sustained release from implants. Delivery by inhalation includes, for example, nasal or oral inhalation, use of a nebulizer, inhalation of the antagonist in aerosol form, and the like. Other alternatives include eyedrops; oral preparations including pills, syrups, lozenges or chewing gum; and topical preparations such as lotions, gels, sprays, and ointments.

Advantageously, IL-23 antibodies are administered in the form of a composition comprising one or more additional components such as a physiologically acceptable carrier, excipient or diluent. Optionally, the composition additionally comprises one or more physiologically active agents for combination therapy. A pharmaceutical composition may comprise an anti-IL-23 antibody together with one or more substances selected from the group consisting of a buffer, an antioxidant such as ascorbic acid, a low molecular weight polypeptide (such as those having fewer than 10 amino acids), a protein, an amino acid, a carbohydrate such as glucose, sucrose or dextrins, a chelating agent such as EDTA, glutathione, a stabilizer, and an excipient. In accordance with appropriate industry standards, preservatives such as benzyl alcohol may also be added. The composition may be formulated as a lyophilizate using appropriate excipient solutions (e.g., sucrose) as diluents. The anti-IL-23 antibody can be provided at a concentration of 50 to 200 mg/ml. Exemplary formulations useful for the present disclosure are those that include a glutamic acid, citric acid or acetic acid buffer at an appropriate pH, from 4.5 to 5.2, an excipient such as sucrose, glycine, proline, glycerol, and/or sorbitol at an appropriate concentration such as 1 to 20% (w/v), and a surfactant such as a non-ionic surfactant like polysorbate (polysorbate 20 or 80) or poloxamers (poloxamer 1888) at an appropriate concentration of 0.001%-0.1% (w/v). Such formulations are disclosed in U.S. Pat. No. 6,171,586 and WIPO Published Applications Nos: WO20100027766 and WO2011088120. In some embodiments, the formulations comprise sodium acetate, sucrose and polysorbate 20. In some embodiments, the formulations comprise 70 mg/mL brazikumab, 10 mM sodium acetate, 9% (w/v) sucrose and 0.004% (w/v) polysorbate 20, at pH 5.2. Suitable components are nontoxic to recipients at the dosages and concentrations employed. Further examples of components that may be employed in pharmaceutical formulations are presented in any edition of Remington's Pharmaceutical Sciences including the 21^{st} Ed. (2005), Mack Publishing Company, Easton, Pa.

Kits for use by medical practitioners include an anti-IL-23 antibody and a label or other instructions for use in treating any of the conditions discussed herein. In one embodiment, the kit includes a sterile preparation of one or more IL-23 antigen-binding proteins, which may be in the form of a composition as disclosed above, and may be in one or more vials.

Particular embodiments of methods of the disclosure involve the use of an anti-IL-23 antibody and one or more additional IL-23 antagonists, as described in U.S. Pat. Nos. 7,491,391; 7,807,414; 7,872,102; 7,807,160; 8,362,212; 7,935,344; 7,790,862; U.S. Published Patent Application Nos. 2012282269, 20090123479; 20120128689; and 2012264917; and WIPO Publications WO1999/05280, WO2007/0244846, WO2007/027714, WO 2007/076524, WO2007/147019, WO2008/103473, WO 2008/103432, WO2009/043933, WO2009/082624 and WO 12/009760.

Also provided are IL-23 antibodies administered alone or in combination with other agents useful for treating ulcerative colitis. Topical medications (e.g., steroids, coal tar, anthralin, Dead Sea salts, various natural oils, vitamin D3 and its analogs, sunshine, topical retinoids), phototherapy (e.g., ultraviolet light, photochemotherapy (PUVA)), and internal medications (e.g., methotrexate, systemic steroids). When multiple therapeutics are co-administered, dosages may be adjusted accordingly, as is recognized or known in the pertinent art.

In every case where a combination of molecules and/or other treatments is used, the individual molecule(s) and/or treatment(s) can be administered in any order, over any length of time that is effective, e.g., simultaneously, consecutively, or alternately. In one embodiment, the method of treatment comprises completing a first course of treatment with one molecule or other treatment before beginning a second course of treatment. The length of time between the end of the first course of treatment and beginning of the second course of treatment can be any length of time that allows the total course of therapy to be effective, e.g., seconds, minutes, hours, days, weeks, months, or even years.

The terms “polypeptide” or “protein” means a macromolecule having the amino acid sequence of a native protein, that is, a protein produced by a naturally occurring and non-recombinant cell; or it is produced by a genetically engineered or recombinant cell, and comprise molecules having the amino acid sequence of the native protein, or molecules having one or more deletions from, insertions to, and/or substitutions of the amino acid residues of the native sequence. The term also includes amino acid polymers in which one or more amino acids are chemical analogs of a corresponding naturally occurring amino acid and polymers. The terms “polypeptide” and “protein” encompass IL-23 antibodies and sequences that have one or more deletions from, additions to, and/or substitutions of the amino acid residues of the antigen-binding protein sequence. The term “polypeptide fragment” refers to a polypeptide that has an amino-terminal deletion, a carboxyl-terminal deletion, and/or an internal deletion as compared with the full-length native protein. Such fragments may also contain modified amino acids as compared with the native protein. In certain embodiments, fragments are about five to 500 amino acids long. For example, fragments may be at least 5, 6, 7, 8, 9, 10, 11, 12, 13, 14, 15, 16, 17, 18, 19, 20, 50, 70, 100, 110, 150, 200, 250, 300, 350, 400, or 450 amino acids long. Useful polypeptide fragments include immunologically functional fragments of antibodies, including binding domains. In the case of an anti-IL-23 antibody, useful fragments include but are not limited to one or more CDR regions, a variable domain of a heavy or light chain, a portion of an antibody chain, a portion of a variable region including less than three CDRs, an Fv, an scFv, a Fab, a Fab′, a F(ab′)2, and the like.

The term “isolated protein” refers to a protein, such as an antigen-binding protein (an example of which could be an antibody), that is purified from proteins or polypeptides or other contaminants that would interfere with its therapeutic, diagnostic, prophylactic, research or other use. As used herein, “substantially pure” means that the described species of molecule is the predominant species present, that is, on a molar basis it is more abundant than any other individual species in the same mixture. In certain embodiments, a substantially pure molecule is a composition wherein the object species comprises at least 50% (on a molar basis) of all macromolecular species present. In other embodiments, a substantially pure composition will comprise at least 80%, 85%, 90%, 95%, or 99% of all macromolecular species present in the composition. In certain embodiments, an essentially homogeneous substance has been purified to such a degree that contaminating species cannot be detected in the composition by conventional detection methods and thus the composition consists of a single detectable macromolecular species.

A “variant” of a polypeptide (e.g., an antigen-binding protein such as an antibody) comprises an amino acid sequence wherein one or more amino acid residues are inserted into, deleted from and/or substituted into the amino acid sequence relative to another polypeptide sequence. Variants include fusion proteins or chimeras. A “derivative” of a polypeptide is a polypeptide that has been chemically modified in some manner distinct from insertion, deletion, or substitution variants, e.g., via conjugation to another chemical moiety. Exemplary protein derivatives are forms of the protein that have been glycosylated, myristoylated, PEGylated, and the like.

The terms “naturally occurring” or “native” as used throughout the specification in connection with biological materials such as polypeptides, nucleic acids, host cells, and the like, refers to materials which are found in nature, such as native human IL-23. In certain aspects, recombinant antigen-binding proteins that bind native IL-23 are provided. In this context, a “recombinant protein” is a protein made using recombinant techniques, i.e., through the expression of a recombinant nucleic acid as described herein. Methods and techniques for the production of recombinant proteins are well known in the art.

The term “antibody” refers to an intact immunoglobulin of any isotype, and of any sub-isotype, or a fragment thereof that can compete with the intact antibody for specific binding to the target antigen, and includes, for instance, chimeric, humanized, fully human, and bispecific antibodies. An antibody as such is a species of an antigen-binding protein. Unless otherwise indicated, the term “antibody” includes, in addition to antibodies comprising two full-length heavy chains and two full-length light chains, derivatives, variants, fragments, and muteins thereof, examples of which are described below. An intact antibody generally will comprise at least two full-length heavy chains and two full-length light chains, but in some instances may include fewer chains such as antibodies naturally occurring in camelids, which may comprise only heavy chains. Antibodies may be derived solely from a single source, or may be “chimeric,” that is, different portions of the antibody may be derived from two different antibodies as described further below. The antigen-binding proteins, antibodies, or binding fragments may be produced in hybridomas, by recombinant DNA techniques, or by enzymatic or chemical cleavage of intact antibodies.

The term “functional fragment” (or simply “fragment”) of an antibody or immunoglobulin chain (heavy or light chain), as used herein, is an antigen-binding protein comprising a portion (regardless of how that portion is obtained or synthesized) of an antibody that lacks at least some of the amino acids present in a full-length chain but which is capable of specifically binding to an antigen. Such fragments are biologically active in that they bind specifically to the target antigen and can compete with other antigen-binding proteins, including intact antibodies, for specific binding to a given epitope. In one aspect, such a fragment will retain at least one complementarity determining region (CDR) present in the full-length light or heavy chain, and in some embodiments will comprise a single heavy chain and/or light chain or portion thereof. These biologically active fragments may be produced by recombinant DNA techniques, or may be produced by enzymatic or chemical cleavage of antigen-binding proteins, including intact antibodies. Fragments include, but are not limited to, immunologically functional fragments such as Fab, Fab′, F(ab′)2, Fv, domain antibodies and single-chain antibodies, and may be derived from any mammalian source, including but not limited to human, mouse, rat, goat, sheep, horse, cow, camelid or rabbit. It is contemplated further that a functional portion of the antigen-binding proteins disclosed herein, for example, one or more CDRs, could be covalently bound to a second protein or to a small molecule to create a therapeutic agent directed to a particular target in the body, possessing bifunctional therapeutic properties, or having a prolonged serum half-life.

An “antigen-binding protein” as used herein means a protein that specifically binds a specified target antigen; the antigen as provided herein is IL-23, particularly human IL-23, including native human IL-23. Antigen-binding proteins as provided herein interact with at least a portion of the unique p19 subunit of IL-23, detectably binding IL-23; but do not bind with any significance to IL-12 (e.g., the p40 and/or the p35 subunits of IL-12). As a consequence, the antigen-binding proteins provided herein are capable of affecting IL-23 activity without the potential risks that inhibition of IL-12 or the shared p40 subunit might incur. The antigen-binding proteins may affect the ability of IL-23 to interact with its receptor, for example by affecting binding to the receptor, such as by interfering with receptor association. In particular, such antigen-binding proteins totally or partially reduce, inhibit, interfere with or modulate one or more biological activities of IL-23. Such inhibition or neutralization disrupts a biological response in the presence of the antigen-binding protein compared to the response in the absence of the antigen-binding protein and can be determined using assays known in the art and described herein. Antigen-binding proteins provided herein inhibit IL-23-induced proinflammatory cytokine production, for example IL-23-induced IL-22 production in whole blood cells and IL-23-induced IFNγ expression in NK and whole blood cells. Reduction of biological activity can be about 20%, 30%, 40%, 50%, 60%, 70%, 80%, 85%, 90%, 91%, 92%, 93%, 94%, 95%, 96%, 97% 98%, 99% or more.

Certain antigen-binding proteins described herein are antibodies, or are derived from antibodies. Such antigen-binding proteins include, but are not limited to, monoclonal antibodies, bispecific antibodies, minibodies, domain antibodies, synthetic antibodies, antibody mimetics, chimeric antibodies, humanized antibodies, human antibodies, antibody fusions, antibody conjugates, single chain antibodies, and fragments thereof, respectively. In some instances, the antigen-binding protein is an immunological fragment of an antibody (e.g., a Fab, a Fab′, a F(ab′)2, or a scFv).

Certain antigen-binding proteins that are provided may comprise one or more CDRs as described herein (e.g., 1, 2, 3, 4, 5, 6 or more CDRs). In some instances, the antigen-binding protein comprises (a) a polypeptide structure and (b) one or more CDRs that are inserted into and/or joined to the polypeptide structure. The polypeptide structure can take a variety of different forms. For example, it can be, or comprise, the framework of a naturally occurring antibody, or fragment or variant thereof, or may be completely synthetic in nature. Examples of various polypeptide structures are further described below.

An antigen-binding protein of the disclosure is said to “specifically bind” its target antigen when the dissociation equilibrium constant (K_{D}) is ≤10⁻⁸ M. The antigen-binding protein specifically binds antigen with “high affinity” when the K_{D} is ≤5×10⁻⁹M, and with “very high affinity” when the K_{D} is ≤5×10⁻¹⁰ M. In one embodiment the antigen-binding protein will bind to human IL-23 with a K_{D} of ≤5×10⁻¹² M, and in yet another embodiment it will bind with a K_{D≤}5×10⁻¹³ M. In another embodiment of the invention, the antigen-binding protein has a K_{D} of ≤5×10⁻¹² M and a Koff of about ≤5×10⁻⁶ 1/s. In another embodiment, the Koff is ≤5×10⁻⁷1/s.

In embodiments where the antigen-binding protein is used for therapeutic applications, an antigen-binding protein can reduce, inhibit, interfere with or modulate one or more biological activities of IL-23, such as by inducing production of proinflammatory cytokines. IL-23 has many distinct biological effects, which can be measured in many different assays in different cell types; examples of such assays are known, see for example U.S. Published Patent Application No: 2013-0004501, the disclosure of which is incorporated by reference herein. Exemplary IL-23 antibodies are disclosed in U.S. Published Patent Application No: 2013-0004501.

As used herein, “brazikumab” (also known as AMG 139) refers to an intact brazikumab immunoglobulin or to an antigen-binding portion thereof that competes with the intact antibody for specific binding, unless otherwise specified. Brazikumab also includes antibodies (or fragments thereof) that are identical or similar to brazikumab in amino acid sequence, particularly in the variable regions, or in the CDRs thereof (however, variations in the constant regions are also contemplated). For example, a useful brazikumab polypeptide has an amino acid sequence that is 85%, 90%, 92%, 95%, 98%, 99% or 100% identical to that of an brazikumab polypeptide disclosed herein. In another embodiment, a useful polypeptide is between 80% 85%, 90%, 92%, 95%, 98%, 99% or 100% identical to brazikumab.

Brazikumab is a human antibody that specifically recognizes the native human IL-23 heterodimer, but does not bind with any significance to the human IL-12 heterodimer. Brazikumab inhibits IL-23-induced proinflammatory cytokine production. For example, IL-23-induced IL-22 production in whole blood cells and IL-23-induced IFNγ expression in NK and whole blood cells. In some embodiments, brazikumab is an isolated, IL-23-specific antigen-binding protein having a heavy chain variable region comprising CDRH1, CDRH2 and CDRH3 from SEQ ID NO:1, and a light chain variable region comprising CDRL1, CDRL2 and CDRL3 from SEQ ID NO:2. In some embodiments, brazikumab is an isolated, IL-23-specific antigen-binding protein wherein the heavy chain variable region is at least 90% identical to SEQ ID NO:1, and the light chain variable region is at least 90% identical to CDRL1, CDRL2 and CDRL3 from SEQ ID NO:2. See, WO 2011/056600, published May 11, 2011.

Where a range of values is provided, it is understood that each intervening value (to the tenth of the unit of the lower limit unless the context clearly dictates otherwise) between the upper and lower limit of that range, and any other stated or intervening value or smaller range in that stated range, is encompassed within the disclosure. The upper and lower limits of smaller ranges may independently be included in the smaller range, subject to any specifically excluded limit in the stated range. Where the stated range includes one or both of the limits, ranges excluding either both of those included limits are also included in the disclosure.

Unless otherwise defined herein, scientific and technical terms used in connection with the disclosure shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art. The methods and techniques of the present invention are generally performed according to conventional methods well-known in the art and as described in various general and more specific references that are cited and discussed throughout the present specification unless otherwise indicated. See, e.g., Sambrook et al., Molecular Cloning: A Laboratory Manual, 3rd ed., Cold Spring Harbor Laboratory Press, Cold Spring Harbor, N.Y. (2001), Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990). Enzymatic reactions and purification techniques are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are those well-known and commonly used in the art. Standard techniques are available for chemical syntheses, chemical analyses, pharmaceutical preparation, formulation, delivery, and treatment of patients.

In preclinical models and studies in patients, anti-IL-12/23 p40 antibodies (e.g., ustekinumab, which is approved for treatment of Crohn's disease and psoriasis, and briakinumab) and anti-IL-23p19 antibodies have been shown to induce clinical responses in Crohn's disease. Brazikumab, previously known as MED12070 and AMG 139, is a human immunoglobulin that selectively binds to human interleukin-23 (IL-23) with high affinity and prevents IL-23 from interacting with the IL-23 receptor. The roles of IL-23 are believed to be important for the recruitment and activation of a range of inflammatory cells involved in inflammation. Brazikumab is a human, Chinese hamster ovary cell-derived, immunoglobulin G2 (IgG2) monoclonal antibody (mAb) consisting of 2 heavy chains of the IgG2 subclass and 2 light chains of the lambda subclass, which are covalently linked through disulfide bonds.

The nonclinical safety of brazikumab was evaluated in several studies with cynomolgus monkeys as the pharmacologically relevant species. In a safety pharmacology study, no brazikumab-related effects were noted on evaluated cardiovascular, respiratory, or neurobehavioral parameters after single intravenous (IV) administration of 300 mg/kg. In studies of 2 weeks, 3 months, and 6 months duration in cynomolgus monkeys, brazikumab was generally well tolerated when administered IV or subcutaneously (SC). Brazikumab administration at doses up to and including 300 mg/kg had no effect on in-life observations, peripheral blood immunophenotyping, or clinical and anatomic pathology, and no sex-related differences in exposure. In the 6-month toxicology study, administration of brazikumab to cynomolgus monkeys by SC injection at 30, 100, or 300 mg/kg once weekly for 26 weeks had no toxicologically significant effects on study parameters. Approximately 14% (4 of 28) of the brazikumab-treated animals developed binding anti-drug antibodies (ADA) during the dosing period and 25% (1 of 4) of animals at 300 mg/kg developed binding ADA in the recovery period. No neutralizing antibodies were detected in animals that tested positive for binding ADA, and binding ADA did not decrease brazikumab exposure. The no observed adverse effect level following 26 weekly SC doses of brazikumab was 300 mg/kg, the maximum dose tested, corresponding to a maximum serum drug concentration (Cₘₐₓ) of 5900 μg/mL and an area under the serum concentration versus time curve (AUC) of 32,100 μg·day/mL on Study Day 176.

In the experiments described in the Examples below, a variety of objectives were sought, and specified endpoints were defined, as disclosed in Table 1.

TABLE 1

| OBJECTIVES | ENDPOINTS |
|---|---|
| PRIMARY | PRIMARY |
| □ TO COMPARE THE EFFICACY OF BRAZIKUMAB | CLINICAL REMISSION: |
| WITH THAT OF PLACEBO TO ACHIEVE CLINICAL | □ MODIFIED MAYO SCORE (MMS) AT WEEK 10: |
| REMISSION | ○ ENDOSCOPY SUBSCORE = 0 OR 1, AND |
| | ○ RECTAL BLEEDING SUBSCORE = 0, AND |
| | ○ STOOL FREQUENCY SUBSCORE = 0 |
| SECONDARY | SECONDARY |
| □ TO COMPARE THE EFFICACY OF BRAZIKUMAB | SUSTAINED CLINICAL REMISSION: |
| WITH THAT OF PLACEBO TO ACHIEVE | □ MMS AT BOTH WEEK 10 AND WEEK 54: |
| SUSTAINED CLINICAL REMISSION | ○ ENDOSCOPY SUBSCORE = 0 OR 1, AND |
| | ○ RECTAL BLEEDING SUBSCORE = 0, AND |
| | ○ STOOL FREQUENCY SUBSCORE = 0 |
| □ TO COMPARE THE EFFICACY OF BRAZIKUMAB | CS-FREE CLINICAL REMISSION: |
| WITH THAT OF PLACEBO TO ACHIEVE | |
| CORTICOSTEROID-FREE (CS-FREE) CLINICAL | □ MMS AT WEEK 54 FOR PARTICIPANTS WHO |
| REMISSION | ARE CS-FREE FOR 12 WEEKS THROUGH |
| | WEEK 54: |
| | ○ ENDOSCOPY SUBSCORE = 0 OR 1, AND |
| | ○ RECTAL BLEEDING SUBSCORE = 0, AND |
| | ○ STOOL FREQUENCY SUBSCORE = 0 |
| □ TO EVALUATE THE PHARMACOKINETICS (PK) | □ POPULATION PK MODEL OF SERUM |
| AND IMMUNOGENICITY OF BRAZIKUMAB IN | CONCENTRATIONS OF BRAZIKUMAB AND |
| PARTICIPANTS WITH UC | ANALYSIS FOR SERUM ANTI-BRAZIKUMAB |
| | ANTIBODIES |
| □ TO CHARACTERIZE THE EXPOSURE- | □ EXPOSURE-RESPONSE MODEL LINKING |
| RESPONSE RELATIONSHIPS OF BRAZIKUMAB | PRIMARY ENDPOINTS TO METRICS OF MODEL- |
| | PREDICTED INDIVIDUAL BRAZIKUMAB |
| | EXPOSURES |
| ADDITIONAL | ADDITIONAL |
| □ TO COMPARE THE EFFICACY OF BRAZIKUMAB | CLINICAL REMISSION: |
| WITH THAT OF VEDOLIZUMAB TO ACHIEVE | □ MMS AT WEEK 10: |
| CLINICAL REMISSION | ○ ENDOSCOPY SUBSCORE = 0 OR 1, AND |
| | ○ RECTAL BLEEDING SUBSCORE = 0, AND |
| | ○ STOOL FREQUENCY SUBSCORE = 0 |
| □ TO COMPARE THE EFFICACY OF BRAZIKUMAB | SUSTAINED CLINICAL REMISSION: |
| WITH THAT OF VEDOLIZUMAB TO ACHIEVE | □ MMS AT BOTH WEEK 10 AND WEEK 54: |
| SUSTAINED CLINICAL REMISSION | ○ ENDOSCOPY SUBSCORE = 0 OR 1, AND |
| | ○ RECTAL BLEEDING SUBSCORE = 0, AND |
| | ○ STOOL FREQUENCY SUBSCORE = 0 |
| □ TO COMPARE THE EFFICACY OF BRAZIKUMAB | CS-FREE CLINICAL REMISSION: |
| WITH THAT OF VEDOLIZUMAB TO ACHIEVE CS- | □ MMS AT WEEK 54 FOR PARTICIPANTS WHO |
| FREE CLINICAL REMISSION | ARE CS-FREE FOR 12 WEEKS THROUGH |
| | WEEK 54: |
| | ○ ENDOSCOPY SUBSCORE = 0 OR 1, AND |
| | ○ RECTAL BLEEDING SUBSCORE = 0, AND |
| | ○ STOOL FREQUENCY SUBSCORE = 0 |
| □ TO EVALUATE THE SAFETY AND | AES, CLINICAL LABORATORY VALUES, VITAL |
| TOLERABILITY OF BRAZIKUMAB IN | SIGNS, PHYSICAL EXAMS, |
| PARTICIPANTS WITH UC | ELECTROCARDIOGRAMS (ECGS) |

The results disclosed in the following Examples establish that administration of brazikumab results in reduced colonic inflammation, which translates into an improved clinical remission rate based on a responder definition that includes stool frequency, rectal bleeding, and endoscopy scores in patients with moderately to severely active UC, including patients who have failed or are intolerant to conventional or biological therapy, or who are naïve to biological therapy, or who have previously received biological agents except for patients who were intolerant to, or had a primary or secondary response to, vedolizumab, as described in Example 3. The Examples are provided for the purpose of illustrating specific embodiments or features of the instant invention and do not limit its scope.

### EXAMPLES

### Example 1

Assays

The assays described in Table 2 are performed according to procedures known in the art and contribute to the results of the experiments disclosed herein.

TABLE 2

| Laboratory | | | | |
|---|---|---|---|---|
| Assessments | | Parameters | | |
| Hematology | Platelet count | RBC indices: | WBC count with differential: | |
| | RBC count | MCV | Neutrophils | |
| | Hemoglobin | MCH | Lymphocytes | |
| | Hematocrit | % Reticulocytes | Monocytes | |
| | | | Eosinophils | |
| | | | Basophils | |
| Clinical Chemistry | BUN | Potassium | Aspartate | Total and direct bilirubin |
| | | | aminotransferase (AST) | |
| | Creatinine | Sodium | Alanine | Total protein |
| | eGRF | | aminotransferase (ALT) | |
| | Glucose (nonfasting) | Calcium | Alkaline phosphatase | Bicarbonate |
| | Magnesium | Chloride | Albumin | Phosphate |
| | Uric acid | | | |

| | |
|---|---|
| Routine Urinalysis^{a} | Specific gravity |
| | pH, glucose, protein, blood, ketones, bilirubin, urobilinogen, nitrite, leukocyte esterase |
| | by dipstick |
| | Microscopic examination (if blood or protein is abnormal) |
| Other Screening | QuantiFERON TB Gold In-Tube |
| Tests | Follicle-stimulating hormone and estradiol (as needed in women of non-childbearing |
| | potential only) |
| | Urine alcohol and drug screen (to include at minimum: amphetamines, barbiturates, |
| | cocaine, opiates, cannabinoids, and benzodiazepines)] |
| | Serum and urine human chorionic gonadotropin (hCG) pregnancy test (as needed for |
| | women of childbearing potential) ^{b} |
| | Serology [(HIV antibody, hepatitis B surface antigen [HBsAg], and hepatitis C virus |
| | antibody)] |
| | All study-required laboratory assessments will be performed by a central laboratory |

^{a}Local urine testing is standard for the protocol unless serum testing is required.

### Pharmacokinetics, Pharmacodynamics and Biomarkers

Venous blood samples are collected for measurement of serum brazikumab concentration. Serum concentration data is analyzed using a population pharmacokinetics (PK) approach and is also used to characterize exposure-response relationships of brazikumab using a population PK model. Pharmacodynamic parameters are determined using conventional techniques known in the art. For serum collection, label the appropriate SST Vacutainer (5 mL size for PK, 15 mL size for ADA, 5 mL for IL-22 and 10 mL for other investigative biomarkers) and cryovials with the coded labels. After blood is drawn, gently invert the tube approximately 5 times to allow the blood to mix with the contents of the tube. Allow blood to clot for 30 minutes at room temperature. After 30 minutes, and within 45 minutes from the time blood is drawn, centrifuge at 1100-1300 g for 15 minutes. Transfer harvested serum immediately (or within 2 hours from blood collection) into at least 2, but up to 7, prechilled, labeled cryovials each for PK, ADA, IL-22 and serum investigative biomarkers. Each vial should contain at least 1 mL of serum. Place the serum tubes in an approximately −70° C. freezer or colder conditions and store in an upright position.

For K₂EDTA plasma collection procedures for LCN2 and other investigative plasma biomarkers, label the appropriate purple-top vacutainer tubes-K₂EDTA (5 mL total volume for LCN2 and 3 mL total volume for other investigative biomarkers) and cryovials with the coded labels. Immediately invert 8-10 times. The plasma must be processed, aliquoted and frozen immediately if possible. If the sample is not able to be processed immediately, it should be processed within 4 hours of obtaining the whole blood sample and kept at 2-8° C. until aliquoted, which should occur within the next six hours. Place the cryovial tubes with plasma samples in an approximately −70° C. freezer or colder and store in an upright position.

PAXgene blood RNA tube collection begins by initially ensuring that the PAXgene Blood RNA Tube is at room temperature (18° C.-25° C.) prior to use and by labeling the appropriate PAXgene blood RNA tubes and cryovials with the coded labels. Draw 2.5 mL whole blood and gently invert the PAXgene Blood RNA Tube 8 to 10 times. Store the PAXgene Blood RNA Tube upright at room temperature (18° C.-25° C.) for a minimum of 2 hours and a maximum of 72 hours before transferring to freezer (−20° C.).

For biomarker evaluations, blood and stool samples are collected and analyzed to evaluate protein, nucleic acid, and cellular biomarkers that relate to brazikumab intervention. Biomarker analyses are designed to elucidate the mechanisms of action of brazikumab, identify subsets of participants responsive to brazikumab, and to characterize a gene signature. Well-known, routine procedures are used for sample collection, processing, storage, and shipment of samples.

Whole blood samples (approximately 2.5 mL) may be collected in PAXgene tubes for total RNA sample preparation. RNA may be used in the analyses of transcript expression using Thermo Fisher Clarion D array and stored for future analyses.

Venous blood samples of approximately 5 mL are collected for measurement of IL-22 serum concentration. Approximately 3 mL of venous blood samples are collected for measurement of K₂EDTA plasma LCN2 concentration. Each serum and plasma sample is divided into two aliquots (one for bioanalysis, and a backup).

A separate set of blood serum/plasma samples (approximately 10 mL of venous blood to obtain a minimum of 5 mL serum per timepoint, and 5 mL whole blood to obtain 2.5 mL plasma) is collected for analysis of circulating soluble factors in relation to inflammatory cell activities. Factors to be analyzed may include, but are not limited to, IFN-γ, IL-6, IL-8, IL-10, IL-12, IL 17A, IL-2, IL-23, and TNFα. Protein analytes are assessed by mass spectrometry or validated immunoassays.

### Example 2

Brazikumab Toxicology Assessment

A randomized, double-blind, double-dummy, active- and placebo-controlled, parallel-group study was designed to study the effects of an IL-23-specific antibody on patients with moderately to severely active ulcerative colitis. Several design features are incorporated in the study to minimize bias, including double-blind and double-dummy techniques and random assignment of participants, helping to ensure that both known and unknown risk factors are distributed evenly between intervention groups. The inclusion of an active-control group as well as a placebo-control group readily reveals whether a failure to distinguish test intervention from placebo implies ineffectiveness of the test intervention or is simply the result of a trial that lacked the ability to identify an active drug. The comparison of placebo to a standard-of-care drug provides internal evidence of assay sensitivity.

Traditional study designs that assess ‘induction of remission’ and ‘maintenance of remission’ as separate studies require that a specific time point is established that defines when induction treatment ends and maintenance treatment begins. Disadvantages of these traditional separate study designs include the potential of selecting an inappropriate timepoint that may not reflect the optimal time at which the pharmacodynamic properties of the treatment occurs, highly variable response rates that encumber estimation of the sample size needed for re-randomization, treatment carryover effects, and difficulties assessing whether the underlying disease process is still active. Typically, participants who demonstrate a clinical response in a traditional induction study are re-randomized into a separate maintenance study and since only responders are allowed to continue, they may not be the most appropriate population to evaluate long-term remission or evaluate participants who may have responded at a later timepoint with continued treatment. However, the current study design makes it possible to evaluate long-term maintenance of remission in participants receiving continuous treatment who have achieved remission at an earlier specified timepoint.

The current study is designed to combine both initial intervention (induction) and maintenance phases into a single study, in a ‘treat-straight-through’ approach. Using this design, participants are randomized to receive induction therapy with study intervention, active control, or placebo and are then treated straight through for the remainder of the study, which includes both an assessment of clinical remission at week 10 and an assessment of sustained remission in participants who were in clinical remission at both week 10 and week 54. The major advantage of this naturalistic design is that it allows evaluation of both induction and maintenance intervention in a single study and avoids some of the complexities noted above that are associated with a traditional re-randomization maintenance design. Also, the consolidation of the benefits of initial intervention can be evaluated with continued intervention, especially for those participants who have responded to the initial intervention but did not meet the remission criteria at week 10, but could be converted to a remitter with continued intervention. This naturalistic design also mimics clinical practice as patients would continue to be treated along a continuum and not have their intervention truncated into an artificially selected time point. Furthermore, preserving the initial randomization assignment to intervention would ensure that long-term maintenance intervention was not biased in favor of participants that achieved remission during the induction period because those who achieved remission with their intervention would still be on the same intervention in the maintenance phase, without any influence of withdrawal or discontinuation of the intervention. Additionally, those who responded to placebo during the induction phase would still be on placebo in the maintenance phase, without any influence of discontinuation of placebo.

Determination of dosages for use in the ulcerative colitis study was aided by recognizing that a single intravenous (IV) dose of 2100 mg brazikumab was well tolerated by healthy white male study participants in a Phase 1 study and multiple 700-mg IV and 210 mg subcutaneous (SC) doses were shown to be well tolerated and efficacious in patients with Crohn's disease in a Phase 2a study. The safety, tolerability, and PK of a single 4200-mg IV dose of brazikumab administered to healthy male and female participants is also assessed to provide dosing information relevant to the UC study disclosed herein. The IV-administered 700-, 1400-, and 2100-mg doses as part of the induction intervention and the SC-administered 210- and 105-mg doses as part of the maintenance intervention in this study are therefore expected to be well-tolerated. Dose-ranging assessments will also be performed during the maintenance intervention period; participants who received IV brazikumab during the induction period will then be randomized 1:1 to receive either 210 mg or 105 mg SC brazikumab every four weeks.

The inclusion of an active-control group as well as a placebo-control group can readily reveal whether a failure to distinguish test intervention from placebo is a result of the ineffectiveness of the test intervention or is simply the result of a trial that lacked the ability to identify an active drug. Participants in the placebo and active comparator groups will undergo the same study assessments as the brazikumab-treated participants.

There are considerable exposure margins between what would be expected from participants in this study receiving multiple IV doses of 2100 mg brazikumab/multiple SC doses of 210 mg brazikumab and animals in the pivotal toxicology studies exposed to brazikumab at the no-observed-adverse-effect-level (NOAEL). The NOAEL of 300 mg/kg for brazikumab was established in cynomolgus monkeys in three studies in which brazikumab was administered intravenously weekly for up to 14 weeks and subcutaneously weekly up to 6 months. At this dose, no toxicologically significant effects were observed. Based on Cₘₐₓ and AUC values in these pivotal toxicology studies in cynomolgus monkeys and comparable PK parameter values from phase 1 and phase 1 b studies in healthy participants, exposure margins were determined, and are presented in Table 3.

TABLE 3
Summary of Brazikumab Cynomolgus Monkey to Human
Margins of Exposure

| | NOAEL | | | Human | |
|---|---|---|---|---|---|
| | Dose | | AUC₀₋ₜₐᵤ | Exposure | |
| | (mg/kg/ | Cₘₐₓ | (μg · | Margins | |
| Study | week) | (μg/mL) | day/mL) | Cₘₐₓ | AUC^{a} |
| 3-Month with 12-month | 300 IV | 14,800 | 53,500 | 8.1^{b} | 11^{b} |
| recovery period (109075) | | | | | |
| 3-Month with 12-month | 300 SC | 7500 | 41,500 | 168^{c} | 172^{c} |
| recovery period (109075) | | | | | |
| 6-Month with 11-week | 300 SC | 5900 | 32,100 | 132^{c} | 133^{c} |
| recovery period (109184) | | | | | |

AUC₀₋ₜₐᵤ = Area Under the serum concentration-time Curve from time zero to the end of the dosing interval;
Cmax = maximum serum concentration; IV = intravenous; SC = subcutaneous.
^{a}The monkey AUC₀₋ₜₐᵤ was multiplied by 4 to adjust for the 28-day dosing interval in humans.
^{b}Safety margins were calculated based on predicted exposures in humans (Cₘₐₓ = 1828 μg/mL; AUC_{0-28day} = 19,250 μg · day/mL) for an IV dose of 4200 mg brazikumab. Predicted exposure estimates were based on human exposures following an IV dose of 2100 mg brazikumab multiplied by 2.
^{c}Safety margins were calculated based on exposures in humans following a SC dose of 210 mg. Human Cₘₐₓ (44.7 μg/mL) and AUC₀₋ₜₐᵤ (967 μg · day/mL) were measured at steady state.

In conclusion, the results from toxicology studies with brazikumab provide data that indicate that brazikumab is considered safe for its intended use in humans.

### Example 3

Brazikumab Treatment of Ulcerative Colitis

Participants in the study are 18 to 80 years of age, inclusive, with moderately to severely active ulcerative colitis who have failed or are intolerant to conventional therapy. This includes participants who have not received a biologic agent (biologic naïve) or have received a biologic agent (e.g., anti-TNFα) at a dose approved for the treatment of UC and did not respond initially (i.e., primary non-response), or responded initially but then lost response with continued therapy (i.e., secondary non-response), or were intolerant to the medication. This also includes patients who have previously received a biologic agent with a successful response without subsequent treatment failure (including vedolizumab). However, since vedolizumab is used as an active comparator, participants who have failed (met the criteria for primary or secondary nonresponse to treatment) or are intolerant to prior treatment with vedolizumab will be excluded.

The inclusion criteria are designed to ensure a patient population that is sufficiently symptomatic to demonstrate a clinically meaningful change from baseline to support a treatment benefit for patients with moderately to severely active ulcerative colitis. The participants are required to have a full colonoscopy within 21 days of randomization to ensure that the appearance of their colonic mucosa is consistent with moderately to severely active UC, to examine and document the extent of colonic surface area that is affected, and to assess if changes in colonic mucosa can reasonably be attributed to the study intervention. Furthermore, the extent of disease assessed by the baseline full colonoscopy may be used to determine if a flexible sigmoidoscopy would be appropriate for the subsequent endoscopic assessments at weeks 10 and 54.

Patients are randomized into five groups, i.e., groups receiving intravenous brazikumab, a group receiving vedolizumab (Entyvio®, an anti-α₄β₇ integrin monoclonal antibody), and a group receiving placebo. Day 1 administrations take place approximately 1 week after randomization of patients, i.e., participants. Table 4 presents details regarding study intervention and administration.

TABLE 4
Study Intervention

| Study | | | | |
|---|---|---|---|---|
| Intervention | Brazikumab | Brazikumab | | |
| Name | IV | SC | Vedolizumab | Placebo^{a} |
| Dosage | Liquid in vial | Liquid in vial | Lyophilized | Liquid in vial |
| Formulation | | | product in vial | |
| Route of | IV infusion | SC | IV infusion | SC and IV |
| Admin- | | | | infusion |
| istration | | | | |
| Dose | 700 mg | 210 mg | 300 mg | 0 |
| Strengths | 1400 mg | 105 mg | | |
| | 2100 mg | | | |
| Dosing | 10 vials for | 3 vials for | 1 vial | SC dosing: 3 |
| Instructions^{b} | 700 mg | 210 mg | | vials |
| | 20 vials for | 1.5 vials for | | IV dosing: 0 |
| | 1400 mg | 105 mg | | vials |
| | 30 vials for | | | |
| | 2100 mg | | | |
| Injection | IV | Needle & | IV | IV |
| Device | admin- | Syringe | admin- | admin- |
| | istration | | istration | istration |
| | set | | set | set |
| | | | | Needle and |
| | | | | Syringe |
| Packaging | Study | Study | Study | SC dosing: |
| and | intervention | intervention | intervention | Study |
| Labeling | will be | will be | will be | intervention |
| | provided in | provided in | provided in | will be |
| | vial. Each | vial. Each | vial. Each | provided in |
| | vial will | vial will | vial will | vial. Each vial |
| | be labeled | be labeled | be labeled | will be labeled |
| | as required | as required | as required | as required |
| | per country | per country | per country | per country |
| | requirement. | requirement. | requirement. | requirement. |
| | | | | IV dosing: |
| | | | | Study |
| | | | | intervention |
| | | | | will not be |
| | | | | provided |
| Manu- | Allergan | Allergan | Takeda | Allergan |
| facturer | | | | |

IV = intravenous; SC = subcutaneous.
^{a}Placebo vials will not be supplied for IV dosing. Instead, unmanipulated IV bags will be provided by an unblinded pharmacist and used as placebo for IV dosing.
^{b}Instructions for the preparation of all doses is provided in the Pharmacy Manual

Brazikumab is administered as a 100 mL IV infusion for the first 3 doses and SC using a standard single-use syringe for all subsequent doses; all vedolizumab doses are administered as a 250 mL IV infusion. Because the preparations of brazikumab and vedolizumab are distinct in appearance and volume, special precautions need to be taken to ensure the double-blind nature of the study. The double-dummy technique is used to maintain the blind when administering the interventions because the brazikumab and vedolizumab interventions cannot be made identical. All participants are administered the same number and type (e.g., IV and/or SC) of interventions throughout the study regardless of intervention group assignment. For example, two IV infusions are administered to each patient on Study Days 1, 15 and 43. The first IV infusion (IV1) is a 100-mL infusion administered over 60 minutes, followed immediately by a second IV 250-mL infusion (IV2) administered over 30 minutes. IV1 must always be administered before IV2. Table 5 presents the by-visit double-dummy administration schedule for the induction period.

TABLE 5
Dosing Regimen for the Induction Period

| | | BRAZIKUMAB | | | | | |
|---|---|---|---|---|---|---|---|
| | | 2100 MG IV | | | | | |
| | | 1400 MG IV | | VEDOLIZUMAB | | PLACEBO | |
| VISIT | | 700 MG IV | | 300 MG IV | | 0 MG | |
| NUMBER | DAY | IV1 | IV2 | IV1 | IV2 | IV1 | IV2 |
| 2 | 1 | A | S | S | A | S | S |
| (BASELINE) | | | | | | | |
| 3 | 15 | A | S | S | A | S | S |
| 5 | 43 | A | S | S | A | S | S |

IV1 = First intravenous dose of 100 ml;
IV2 = Second intravenous dose of 250 ml;
A = Active;
S = Sham placebo

For the double-blind maintenance period, all participants receive one 250-mL IV infusion administered over 30 minutes per the dosing schedule (e.g., every 8 weeks). In addition, all participants receive 3 separate 1 mL SC injections using a standard single-use SC syringe per the dosing schedule (e.g., every 4 weeks). Table 6 presents the by-visit double-dummy administration schedule for the maintenance period.

TABLE 6
Dosing Regimen for the Maintenance Period

| | | BRAZIKUMAB | | | | | |
|---|---|---|---|---|---|---|---|
| | | 210 MG SC | | VEDOLIZUMAB | | PLACEBO | |
| VISIT | | 105 MG SC | | 300 MG IV | | 0 | |
| NUMBER | DAY | IV | SC | IV | SC | IV | SC |
| 6 | 71 | — | A | — | S | — | S |
| 7 | 99 | S | A | A | S | S | S |
| 8 | 127 | — | A | — | S | — | S |
| 9 | 155 | S | A | A | S | S | S |
| 10 | 183 | — | A | — | S | — | S |
| 11 | 211 | S | A | A | S | S | S |
| 12 | 239 | — | A | — | S | — | S |
| 13 | 267 | S | A | A | S | S | S |
| 14 | 295 | — | A | — | S | — | S |
| 15 | 323 | S | A | A | S | S | S |
| 16 | 351 | — | A | — | S | — | S |

All study interventions are prepared by an unblinded pharmacist (or appropriately qualified individual) and delivered to qualified site staff who will administer the study intervention to participants. The unblinded pharmacist is responsible for preparing the double dummy IV and SC doses according to the Pharmacy Manual. An independent study intervention monitor is also unblinded to perform study intervention accountability.

All Participants receive 2 IV infusions of study intervention (brazikumab, vedolizumab, or placebo) on Days 1, 15, and 43 (Visits 2, 3, and 5) of the induction period. An experienced and qualified staff member places the IV access.

IV1 (brazikumab or placebo) is delivered in 5% weight/volume (w/v) dextrose in water in a volume of 100 mL over a minimum of 60 minutes using an infusion pump. Before and after each IV1 infusion, the IV access is flushed with 30 mL of 5% w/v dextrose in water.

IV2 (vedolizumab or placebo) is delivered in sterile 0.9% sodium chloride in a volume of 250 mL over a minimum of 30 minutes using an infusion pump. Before and after each IV2 infusion, the IV access is flushed with 30 mL of sterile 0.9% sodium chloride injection.

For the maintenance period, all participants receive only one IV infusion of study intervention at the visits specified in Table 5. The IV infusion during the maintenance period (vedolizumab or placebo) is delivered in sterile 0.9% sodium chloride in a volume of 250 mL over a minimum of 30 minutes using an infusion pump. Before and after each IV infusion, the IV access is flushed with 30 mL of sterile 0.9% sodium chloride injection.

The disclosure contemplates exemplary doses of about 120 mg/ml to deliver about 700, 720, 1400, 1440, 2100, 2180, or 4200 mg of brazikumab intravenously during the induction period, and about 120 or 240 mg of brazikumab delivered subcutaneously every four weeks for the maintenance period. Another exemplary dosage is the delivery of about 240 mg of brazikumab delivered subcutaneously every eight weeks during the maintenance period following the three intravenous doses delivered during the induction period.

Vital signs (blood pressure [BP], temperature, pulse rate, and respiration rate) are obtained before IV study intervention administration at all treatment visits. In addition, participants are monitored for changes in vital signs and/or new symptoms approximately every 15 minutes during IV administration, immediately after completion of infusion, and at approximately every 30 minutes for a minimum of one-hour post-infusion or until stable, whichever is later. The participant is discharged from the site when they are deemed clinically stable by the investigator, a minimum of one hour after completion of IV administration for the initial 3 infusions (Visits 2, 3, and 5).

Brazikumab or placebo is also administered to all participants during the maintenance period by SC injection at the visits specified in Table 5. The SC injection is administered after IV administration to maintain consistency in procedures. Each SC dose is administered to the participants anterior abdominal wall. Each SC injection is no more than 1.0 mL in volume (i.e., 3×1.0 mL injections to be administered for all SC doses). The brazikumab or placebo dose is equally divided in 3 syringes and administered as multiple SC injections on alternating (left or right) sites on a participant's anterior abdominal wall over no more than 10 minutes total time for all SC injections and at a distance of at least 2 cm apart. Brazikumab or placebo is slowly injected (at least a 5-second duration is recommended) into the SC tissue using gentle pressure. The area should not be massaged after injection. Injection sites should be rotated.

Vital signs (BP, temperature, pulse rate, and respiration rate) are obtained before and immediately after SC study intervention administration during all treatment visits. In addition, for Visit 6 and Visit 7 (first two SC doses) participants are monitored for changes in vital signs and/or new symptoms approximately every 30 minutes for a minimum of one hour post-injection or until stable, whichever is longer. For the third and subsequent SC doses of brazikumab or placebo, participants are monitored for a minimum of 30 minutes or until stable, whichever is longer.

Infusion reactions have been reported with the administration of IV monoclonal antibodies (mAbs), and reactions to SC administration have also been reported. As with any antibody, allergic reactions to dose administration are possible, and appropriate precautions are taken to address any such reactions.

Efficacy is assessed using the Mayo Scoring System for assessment of UC (Mayo score) as described by Schroeder et al, N. Engl. J. Med. 317:1625-1629 (1987). The Mayo score assesses stool frequency, rectal bleeding, endoscopic findings, and the physician's assessment of disease activity. The Mayo score has been modified to specify no friability in the endoscopy subscore of 1 (mild disease). Stool frequency and rectal bleeding is assessed daily by the patient. The Mayo item for stool frequency has been operationalized as bowel movement frequency to improve clarity and facilitate consistent interpretation of this item among participants. The calculation of the stool frequency and rectal bleeding scores for eligibility is based on the participant's nightly diary data recorded over the most recent three-days within five days before the initiation of the bowel preparation for the screening colonoscopy. The calculation of the stool frequency and rectal bleeding scores during the study at Visits 6, 11, and 17 is based on the participant's nightly diary data recorded over the three prior consecutive days before the initiation of the bowel preparation for the endoscopy.

The mucosal appearance during the endoscopic examination is assessed for the modified Mayo endoscopic subscore (i.e., findings of endoscopy item). The endoscopic appearance is examined by both the investigator and the central reader. Centrally read endoscopic subscores are used for all eligibility and efficacy analyses.

The physician's global assessment (PGA) acknowledges the participant's symptoms, as well as the participant's general sense of well-being, and other observations, such as endoscopic findings. The endoscopic subscore and the PGA are performed by a physician qualified to perform endoscopy, and it is recommended that the same physician perform all assessments for a particular participant throughout the course of the study. The PGA is only used as an exploratory endpoint.

Additional efficacy measures are also contemplated. Supplementing or complementing the primary efficacy measure provided by the modified Mayo Test (Mayo Scoring System) are the Geboes Historical Index (GBI), the Robarts Histopathology Index (RHI), the Functional Assessment of Chronic Illness Therapy—Fatigue (FACIT-F), the Patient Global Impression of Change—Ulcerative Colitis (PGIC-UC), the Patient Global Impression of Severity—Ulcerative Colitis (PGIS-UC), the Patient Impression of Severity—Rectal Bleeding (PIS-RB), the Patient Impression of Interference—Bowel Movement Frequency (PII-BMF), the Inflammatory Bowel Disease Questionnaire (IBDQ), the 12-Item Short Form Survey (SF-12), the 5-Level EuroQoL-5D (EQ-5D-5L), and the Cochran-Mantel-Haenszel Test (CMH Test).

Planned efficacy assessments are shown by timepoint in Table 7. The Mayo scoring system is shown in Table 8. Data for the primary and secondary efficacy assessments and diary data are collected via e diary.

TABLE 7
Planned Efficacy Assessments by Timepoint

| Assessment | Timing | Measurement |
|---|---|---|
| Mayo score items: | | |
| Stool frequency | Visit 1 through | Each individual item |
| | Follow-up 2 (e- | assessed on 0-3 ordinal |
| | diary) | scale described in |
| Rectal bleeding | Visit 1 through | Table |
| | Follow-up 2 (e- | |
| | diary) | |
| Findings of endoscopy | Visits 1, 6, 17 | |
| Physician's global assessment^{a} | Visits 1, 6, 17 | |

^{a}Exploratory endpoint only

TABLE 8
Mayo Scoring System for Assessment of Ulcerative Colitis Activity
Stool Frequency^{a}
0 = Normal number of bowel movements for this participant
1 = 1 to 2 bowel movements more than normal
2 = 3 to 4 bowel movements more than normal
3 = 5 or more bowel movements more than normal
Rectal Bleeding^{b}
0 = No blood seen
1 = Streaks of blood with stool less than half the time
2 = Obvious blood with stool most of the time
3 = Blood alone passed
Findings of Endoscopy^{c}
0 = Normal or inactive disease
1 = Mild disease (erythema, decreased vascular pattern, no friability)
2 = Moderate disease (marked erythema, absent vascular pattern,
friability, erosions)
3 = Severe disease (spontaneous bleeding, ulceration)
Physicians' Global Assessment
0 = Normal
1 = Mild disease
2 = Moderate disease
3 = Severe disease
^{a}Each participant serves as his or her own control to establish the degree of abnormality of the stool frequency.
^{b}The daily bleeding score represented the most severe bleeding of the day.
^{c}Subscore of 1 (mild disease) modified to reflect no friability for modified Mayo Score as described above.
Source: Amended from Schroeder 1987.

A full colonoscopy is required at baseline for all participants. All remaining endoscopic assessments during the study can be performed via a flexible sigmoidoscopy, as clinically indicated. All colonoscopy or flexible sigmoidoscopy procedures for the assessment of endoscopic findings for the mMS may be recorded using video capture. All video recordings are labeled with segment names by the central reader vendor to produce a complete colonoscopy video. The video clips are read centrally for endoscopic severity based on the modified Mayo endoscopy score by independent gastroenterologists experienced in IBD who are blinded to the participants' clinical activity and intervention allocation. In all cases, video recordings should be performed prior to biopsy.

Mucosal biopsies are collected at each study endoscopy (Visits 1, 6, and 17 and/or Early Termination Visit). The biopsies will be used to support assessments of changes over time in the Geboes Histological Index (GHI), the Robarts Histopathology Index (RHI), and the cellular composition of the inflammatory infiltrate including, but not limited to, eosinophils and neutrophils.

The GHI is a measure of the degree of mucosal inflammation as follows: 0=structural change only; 1=chronic inflammation; 2=lamina propria neutrophils; 3=neutrophils in epithelium; 4=crypt destruction; and 5=erosions or ulcers. The biopsies that are obtained during the endoscopy are scored according to the GHI.

The RHI incorporates four histological descriptors (severity of chronic inflammatory infiltrate, the number of lamina propria neutrophils, the number of neutrophils in the epithelium, and the severity of erosions or ulceration), each of which is objectively graded between 0 and 3 (Mosli et al., Gut 66:50-58 (2017). The RHI is calculated based on the information from the GHI.

Information is captured by patients on an event-driven basis after each bowel movement, which is defined as a trip to the toilet when the patient either passes a stool, blood alone, blood and mucus, or mucus only. Participants are instructed to record the occurrence of each bowel movement, presence of stool, blood, and/or mucus in the bowel movement, stool consistency using the Bristol Stool Form Scale (BSFS), and feeling of urgency prior to their bowel movement.

In addition to the event-driven assessments, participants are instructed to complete a nightly diary. The nightly diary consists of the Mayo Score stool frequency and rectal bleeding items, and daily recall items that measure other salient signs and symptoms of UC (fatigue, tiredness, weakness, lack of energy, abdominal pain, frequency and severity of flatulence, and pain associated with sore joints). At the end of each week, the nightly diary will include the Functional Assessment of Chronic Illness Therapy—Fatigue (FACIT-F), the Patient Global Impression of Severity-Ulcerative Colitis (PGIS-UC), Patient Impression of Severity—Rectal Bleeding (PIS-RB), Patient Impression of Interference—Bowel Movement Frequency (PII-BMF), and the Patient Global Impression of Change-Ulcerative Colitis (PGIC-UC). The nightly diary is prompted every evening during the screening and induction period. During the maintenance period, nightly diaries are entered every evening during the week prior to each visit.

The FACIT-F Scale (Version 4) is a 13-item instrument that measures fatigue and its impact on daily functions over a recall period of seven days. Five of the items assess the experience of fatigue and eight items assess the impact of fatigue. Items are scored on a 5-point Likert scale, yielding a score ranging from 0 to 52, with lower scores indicating greater fatigue. FACIT-F has been widely used in clinical trials and with participants with IBD (Tinsley 2011). FACIT-F is designed to be self-administered and can be completed in under 5 minutes. Patient Global Impression of Severity-Ulcerative Colitis (PGIS-UC) is a single item that assesses participants' perceptions of overall severity of UC symptoms for the last 7 days with response options ranging from “none” to “severe.” Patient Impression of Severity-Rectal Bleeding (PIS-RB) is a single item that assesses participants' perceptions of overall severity of rectal bleeding for the last 7 days with response options ranging from “none” to “severe.” Patient Impression of Interference—Bowel Movement Frequency (PII-BMF) is a single item that assesses participants' perceptions of the level of interference in activities of daily living due to frequent bowel movement for the last 7 days with response options ranging from “never” to “always.” Patient Global Impression of Change-Ulcerative Colitis (PGIC-UC) is a single item that measures participants' perceptions of overall change in their UC symptoms over the last 7 days. Additional assessments will involve site visits to obtain patient-reported outcomes (PROs) and participants will be asked to complete questionnaires, such as the Inflammatory Bowel Disease Questionnaire (IBDQ). Throughout the study, participants are monitored for adverse events to ensure their safety.

### REFERENCES

Bettelli E, Korn T, Kuchroo V K. Th17: the third member of the effector T cell trilogy. Curr Opin Immunol. 2007; 19:652-657.
Brazikumab (MED12070) Investigator's Brochure, 2018; Edition 4.0.
Burton P R, Clayton D G, Cardon L R, Craddock N, Deloukas P, Duncanson A, et al. Association scan of 14,500 nonsynonymous SNPs in four diseases identifies autoimmunity variants. Nature Genet. 2007; 39:1329-1337.
Cargill M, Schrodi S J, Chang M, Garcia V E, Brandon R, Callis K P, et al. A large-scale genetic association study confirms IL12B and leads to the identification of IL23R as psoriasis-risk genes. Am J of Human Gen. 2007; 80:273-290.
Duerr R H, Taylor K_{D}, Brant S R, Rioux J D, Silverberg M S, Daly M J, et al. A genome-wide association study identifies IL23R as an inflammatory bowel disease gene. Science 2006; 314:1461-1463.
Entyvio® Package Insert (2014).
Farago B, Magyari L, Sáfrány E, Csöngei V, Járomi L, Horvatovich K, et al. Functional variants of interleukin-23 receptor gene confer risk for rheumatoid arthritis but not for systemic sclerosis. Ann Rheum Dis. 2008; 67:248-250.
Feagan B G, Sandborn W J, Gasink C, Jacobstein D, Lang Y, Friedman J R, et al. Ustekinumab as induction and maintenance therapy for Crohn's disease. N Engl J Med. 2016; 375:1946-1960.
Feagan B G, Sandborn W J, D'Haens G, Panes J, Kaser A, Ferrante M, et al. Induction therapy with the selective interleukin-23 inhibitor risankizumab in patients with moderate-to-severe Crohn's disease: a randomised, double-blind, placebo-controlled phase 2 study. Lancet. 2017; 389:1699-1709.
Gordon K, Langley R, Gottlieb, Papp K A, Krueger G G, Strober B E, et al. A phase III, randomized, controlled trial of the fully human IL-12/23 mAb briakinumab in moderate-to-severe psoriasis. J Invest Dermatol. 2012; 132:304-314.
Gottlieb A B; Leonardi C, Kerdel F, Mehlis S, Olds M, Williams D A. Efficacy and safety of briakinumab vs. etanercept and placebo in patients with moderate to severe chronic plaque psoriasis. Br J Dermatol. 2011; 165:652-660.
Guyatt G, Mitchell A, Irvine E J, Singer J, Williams N et al. A new measure of health status for clinical trials in inflammatory bowel disease. Gastroenterology. 1989; 96:804-810.
Illes Z, Safrany E, Peterfalvi A, Magyari L, Farago B, Pozsonyi E, et al. 3′UTR C2370A allele of the IL-23 receptor gene is associated with relapsing-remitting multiple sclerosis. Neurosci Lett. 2008; 431:36-38.
Kimball A B, Papp K A, Wasfi Y, Chan D, Bissonnette R, Sofen H, et al. Long-term efficacy and safety of ustekinumab in patients with moderate to severe psoriasis through 5 years of follow-up: results from the PHOENIX 1 long-term extension. Br J Dermatol. 2012; 167(Suppl. 1):64 (Abstract P94).
Langley R G, Williams D, Papp K, Olds M. Long-term safety and efficacy of ABT-874 for the treatment of moderate to severe psoriasis: Interim analysis from an open-label extension study. J Am Acad Dermatol. 2012; 66:AB195 (Abstract 4779).
Lee E, Trepicchio W L, Oestreicher J L, Pittman D, Wang F, Chamian F, et al. Increased expression of interleukin 23 p19 and p40 in lesional skin of patients with psoriasis vulgaris. J Exp Med. 2004; 199:125-130.
Leonardi C L, Kimball A B, Papp K A, Yeilding N, Guzzo C, Wang Y, et al. Efficacy and safety of ustekinumab, a human interleukin-12/23 monoclonal antibody, in patients with psoriasis: 76 week results from a randomised, double-blind, placebo-controlled trial (PHOENIX 1). Lancet. 2008; 371:1665-1674.
Li Y, Chu N, Hu A, Gran B, Rostami A, Zhang G X. Increased IL-23p19 expression in multiple sclerosis lesions and its induction in microglia. Brain. 2007; 130:490-501.
Mannon P J, Fuss I J, Mayer L, Elson C O, Sandborn W J, Present D, et al. Anti-interleukin 12 antibody for active Crohn's disease. N Eng J Med. 2004; 351:2069 2079.
Mosli M H, Feagan B G, Zou G, Sandborn W J, D'Haens G, Khanna R, et al. Development and validation of a histological index for U C. Gut 2017; 66:50-58.
Papp K A, Langley R G, Lebwohl M, Krueger G G, Szapary P, Yeilding N, et al. Efficacy and safety of ustekinumab, a human interleukin-12/23 monoclonal antibody, in patients with psoriasis: 52-week results from a randomised, double-blind, placebo-controlled trial (PHOENIX 2). Lancet. 2008; 371:1675-1684.
Reich K, Langley R G, Papp K A, Ortonne J-P, Unnebrink K, Kaul M, et al. A 52-week trial comparing briakinumab with methotrexate in patients with psoriasis. N Engl J Med. 2011; 365:1586-1596.
Sandborn W J, Gasink C, Gao L L, Blank M A, Johanns J, Guzzo C, et al. Ustekinumab induction and maintenance therapy in refractory Crohn's disease. N Engl J Med. 2012; 367:1519-1528.
Sands B E, Chen J, Feagan B G, Penney M, Rees W A, Danese S, et al. Efficacy and safety of MED12070, an antibody against interleukin 23, in patients with moderate to severe Crohn's disease: a phase 2a study. Gastroenterol. 2017; 153:77-86.
Schmidt C, Giese T, Ludwig B, Mueller-Molaian I, Marth T, Zeuzem S, et al. Expression of interleukin-12-related cytokine transcripts in inflammatory bowel disease: elevated interleukin-23p19 and interleukin-27p28 in Crohn's disease but not in ulcerative colitis. Inflamm Bowel Dis. 2005; 11:16-23.
Schroeder K W, Tremaine W J, Ilstrup D M. Coated oral 5-aminosalicylic acid 548 therapy for mildly to moderately active ulcerative colitis. N Engl J Med. 1987; 317:1625-1629.
Sofen H, Smith S, Matheson R, Leonardi C, Calderon C, Bouman E, et al. Results of a single ascending dose study to assess the safety and tolerability of CNTO 1959 following intravenous or subcutaneous administration in healthy subjects and in subjects with moderate to severe psoriasis. Br J Dermatol. 2011; 165:e10 (Abstract FC-21).
Strober B E, Crowley J J, Yamauchi P S, Olds M, Williams D A. Efficacy and safety results from a phase III, randomized controlled trial comparing the safety and efficacy of briakinumab with etanercept and placebo in patients with moderate to severe chronic plaque psoriasis. Br J Dermatol. 2011; 165:661-668.
Tinsley A, Macklin E A, Korzenik J R, Sands B E. Validation of the functional assessment of chronic illness therapy-fatigue (FACIT-F) in patients with inflammatory bowel disease. Aliment Pharmacol Ther. 2011; 34:1328-1336.
Vaknin-Dembinsky A, Balashov K, Weiner H L. IL-23 is increased in dendritic cells in multiple sclerosis and down regulation of IL-23 by antisense oligos increases dendritic cell IL 10 production. J of Immunol. 2006; 176:7768-7774.

All patents and other publications identified are expressly incorporated herein by reference in their entirety for the purpose of describing and disclosing, for example, the methodologies described in such publications that might be used in connection with information described herein.

What is claimed is:

1. A method of treating ulcerative colitis in a subject comprising administering a therapeutically effective amount of an anti-IL-23 antibody that does not inhibit IL-12 to a subject with ulcerative colitis.

2. The method of claim 1 wherein the subject has moderately to severely active ulcerative colitis as determined by a colonoscopy.

3. The method of claim 1 wherein the anti-IL-23 antibody is administered by intravenous infusion.

4. The method of claim 3 wherein a total dosage of at least 700 mg, at least 1400 mg, at least 2100 mg, or at least 4200 mg of anti-IL-23 antibody is administered.

5. The method of claim 3 wherein the intravenous infusion comprises at least 70 mg of anti-IL-23 antibody in a volume of about 100 ml delivered over a period of at least 30 minutes.

6. The method of claim 1 wherein a plurality of intravenous infusions is administered.

7. The method of claim 6 wherein the plurality of intravenous infusions each comprise the same quantity of anti-IL-23 antibody.

8. The method of claim 1 wherein the anti-IL-23 antibody is administered subcutaneously.

9. The method of claim 8 wherein the anti-IL-23 antibody is administered in a plurality of doses.

10. The method of claim 9 wherein a total dosage of at least 105 mg or at least 210 mg of anti-IL-23 antibody is administered.

11. The method of claim 9 wherein each dose comprises about 70 mg anti-IL-23 antibody.

12. The method of claim 1 further comprising a plurality of doses of anti-IL-23 antibody, wherein a second dose is administered about two weeks after a first dose, and a third and subsequent doses are administered about four weeks after a preceding dose.

13. The method of claim 12 wherein the plurality of doses is about 10 doses.

14. The method of claim 12 wherein the first and second doses are administered by intravenous infusion and any subsequent dose is administered subcutaneously.

15. The method of claim 13 wherein each dose comprises at least 70 mg of anti-IL-23 antibody.

16. The method of claim 1 further comprising measuring the effect of the therapy using the modified Mayo Score/Disease Activity Index for Ulcerative Colitis.

17. The method of claim 16 wherein the therapy lowers the score of at least two components of the modified Mayo Score/Disease Activity Index for Ulcerative Colitis, wherein the components are selected from the group consisting of Stool Frequency, Rectal Bleeding, Endoscopy Findings and Physicians' Global Assessment.

18. The method of claim 1 wherein the anti-IL-23 antibody comprises the CDRH1 of SEQ ID NO:3, the CDRH2 of SEQ ID NO:4, the CDRH3 of SEQ ID NO:5, the CDRL1 of SEQ ID NO:6, the CDRL2 of SEQ ID NO:7 and the CDRL3 of SEQ ID NO:8.

19. The method of claim 1 wherein the anti-IL-23 antibody comprises the heavy chain variable region sequence of SEQ ID NO:1.

20. The method of claim 1 wherein the anti-IL-23 antibody comprises the light chain variable region sequence of SEQ ID NO:2.

21. The method of claim 1 wherein the anti-IL-23 antibody comprises the heavy chain variable region sequence of SEQ ID NO:1 and the light chain variable region sequence of SEQ ID NO:2.


SEQUENCE LISTING (8 sequences)

SEQ ID NO: 1 - protein, 124 aa, Artificial Sequence
  Synthetic Polypeptide
  MISC_FEATURE: Brazikumab heavy chain variable region amino acid sequence
  MISC_FEATURE (31)..(35): CDRH1
  MISC_FEATURE (50)..(67): CDRH2
  MISC_FEATURE (99)..(113): CDRH3
QVQLVESGGGVVQPGRSLRLSCAASGFTFSSYGMHWVRQAPGKGLEWVAVIWYDGSNEYYADSVKGRFTISRDNSKNTLYLQMNSLRAEDTAVYYCARDRGYTSSWYPDAFDIWGQGTMVTVSS

SEQ ID NO: 2 - protein, 111 aa, Artificial Sequence
  Synthetic Polypeptide
  MISC_FEATURE: Brazikumab light chain variable region amino acid sequence
  MISC_FEATURE (23)..(36): CDRL1
  MISC_FEATURE (52)..(58): CDRL2
  MISC_FEATURE (91)..(101): CDRL3
QSVLTQPPSVSGAPGQRVTISCTGSSSNTGAGYDVHWYQQVPGTAPKLLIYGSGNRPSGVPDRFSGSKSGTSASLAITGLQAEDEADYYCQSYDSSLSGWVFGGGTRLTVL

SEQ ID NO: 3 - protein, 5 aa, Artificial Sequence
  Synthetic Polypeptide
  MISC_FEATURE: Brazikumab CDRH1 amino acid sequence
SYGMH

SEQ ID NO: 4 - protein, 18 aa, Artificial Sequence
  Synthetic Polypeptide
  MISC_FEATURE: Brazikumab CDRH2 amino acid sequence
VIWYDGSNEYYADSVKGR

SEQ ID NO: 5 - protein, 15 aa, Artificial Sequence
  Synthetic Polypeptide
  MISC_FEATURE: Brazikumab CDRH3 amino acid sequence
DRGYTSSWYPDAFDI

SEQ ID NO: 6 - protein, 14 aa, Artificial Sequence
  Synthetic Polypeptide
  MISC_FEATURE: Brazikumab CDRL1 amino acid sequence
TGSSSNTGAGYDVH

SEQ ID NO: 7 - protein, 7 aa, Artificial Sequence
  Synthetic Polypeptide
  MISC_FEATURE: Brazikumab CDRL2 amino acid sequence
GSGNRPS

SEQ ID NO: 8 - protein, 11 aa, Artificial Sequence
  Synthetic Polypeptide
  MISC_FEATURE: Brazikumab CDRL3 amino acid sequence
QSYDSSLSGWV